(19) United States
(12) Patent Application Publication
(10) Pub. No.: US 2003/0044514 A1
(43) Pub. Date: Mar. 6, 2003

(54) Using supercritical fluids to infuse therapeutic on a medical device

(76) Inventor: Robert E. Richard, Wrentham, MA (US)

Correspondence Address: KENYON & KENYON, 1500 K STREET, N.W., SUITE 700, WASHINGTON, DC, 20005, US

(21) Appl. No.: 09/879,216

(22) Filed: Jun. 13, 2001

(51) Int. Cl. B05D 3/04; A61L 2/00; A61F 2/06

(52) U.S. Cl. 427/2.1; 427/337; 623/1.42; 604/48

(57) ABSTRACT

A method of coating a medical device is provided. The method includes interfacing a therapeutic with a supercritical fluid and transferring the therapeutic from the supercritical fluid that has been interfaced with the therapeutic to the medical device. An article of manufacture is also provided. The article of manufacture includes a medical device having a releasable therapeutic on one of its surfaces, the medical device manufactured by placing the medical device in a coating chamber and exposing the medical device to a supercritical fluid carrying a therapeutic.

### TECHNICAL FIELD

The present invention regards method and apparatus for applying a material to a surface of a work-piece. More particularly the present invention regards the use of supercritical fluids to infuse a therapeutic on a medical device.

### BACKGROUND

A supercritical fluid is any substance above its critical temperature and critical pressure. When a substance is placed above these two points, it enters into its “supercritical range.” While in this range the supercritical fluid exhibits both gas-like and liquid-like properties. Its density may be similar to that of a very dense gas, its diffusivity may be similar to diffusivities normally associated with gases, and its solubility may be similar to that of a liquid. Supercritical fluids will exhibit these properties as long as these are maintained in their supercritical range. When, however, either the temperature or the pressure of a supercritical fluid drops below its associated critical point the fluid will no longer be classified as a supercritical fluid because it will no longer posses some or all of the mixed property characteristics associated with a substance in this range.

Supercritical fluids have been used in various applications including food processing and parts cleaning. Their high solubilities and diffusivities make them an attractive choice for these applications.

Carbon dioxide, is one example of a substance that may be manipulated and placed into its supercritical range. Carbon dioxide is an attractive choice for use as a supercritical fluids. It is an abundant non-toxic material that exhibits a high level of solubility when placed in this supercritical range.

The in-situ delivery of therapeutic within a body of a patient is common in the practice of modern medicine. This in-situ delivery is often completed with coated medical devices that may be temporarily or permanently placed at a target site within the body. These medical devices can be maintained, as required, at their target sites for short and prolonged periods of time, in order to deliver therapeutic to the target site. These medical devices may be coated with a therapeutic or a combination of a therapeutic and a carrier material. Once placed within the body, the therapeutic may be released from the medical device into the target area and, thus, may be able to treat the targeted area. Examples of medical devices that may be coated with therapeutic for delivery to a target site include: expandable and self-expanding stents, balloon catheters, vena-cava filters, aneurysm coils, stent-grafts, a-v shunts, angio-catheters, and PICCs (Peripherally-Inserted Central Catheters).

### SUMMARY OF THE INVENTION

A method of coating a medical device is provided. This method includes interfacing a therapeutic with a supercritical fluid and transferring the therapeutic from the supercritical fluid to the medical device.

An article of manufacture is also provided. This article of manufacture may include a medical device having a releasable therapeutic on one of its surfaces wherein the medical device may be manufactured by placing it in a coating chamber and exposing it to a supercritical fluid carrying a therapeutic.

### BRIEF DESCRIPTION OF THE DRAWINGS

FIG. 1 is a temperature and pressure graph of an exemplary material illustrating its supercritical range.

FIG. 2 is a side view of a coating system in accord with one embodiment of the present invention.

FIG. 3 is a side view of a coating system in accord with an alternative embodiment of the present invention.

FIG. 4 is a side view of a coating system in accord with another alternative embodiment of the present invention.

### DETAILED DESCRIPTION

FIG. 1 is a temperature and pressure graph 10 of an exemplary material illustrating the critical point 17 and the supercritical range 16 (delineated by rays 18 and 19) in which it behaves as a supercritical fluid. The graph 10 in FIG. 1 has temperature graphed along its x-axis 12 and pressure graphed along it y-axis 11. The material graphed in graph 10 behaves as a liquid (i.e., its in a liquid state) when its temperature and pressure are above line 13, as would be the case with point 15. Likewise the material behaves as a gas (i.e., its in a gaseous state) when its temperature and pressure correlate to a point below line 13, as would be the case with point 14. Thus, the definition line 13 separates these two defined states of matter.

When the temperature and pressure of the material reach the critical point 17 the material becomes a supercritical fluid and will remain as such as long as the temperature and pressure exist within the supercritical range 16. Should the pressure exerted on the material drop below the critical point the material will behave as a gas. Similarly, should the temperature drop below the critical point the material will behave as a liquid.

Critical temperatures and critical pressures are not universal thresholds but may, instead, vary from material to material. Moreover, they may require extreme temperatures and pressures, reaching hundreds of degrees Celsius and thousands of atmospheres of pressure in some materials, while for others, like carbon dioxide, they may require less extreme and, therefore, more manageable conditions.

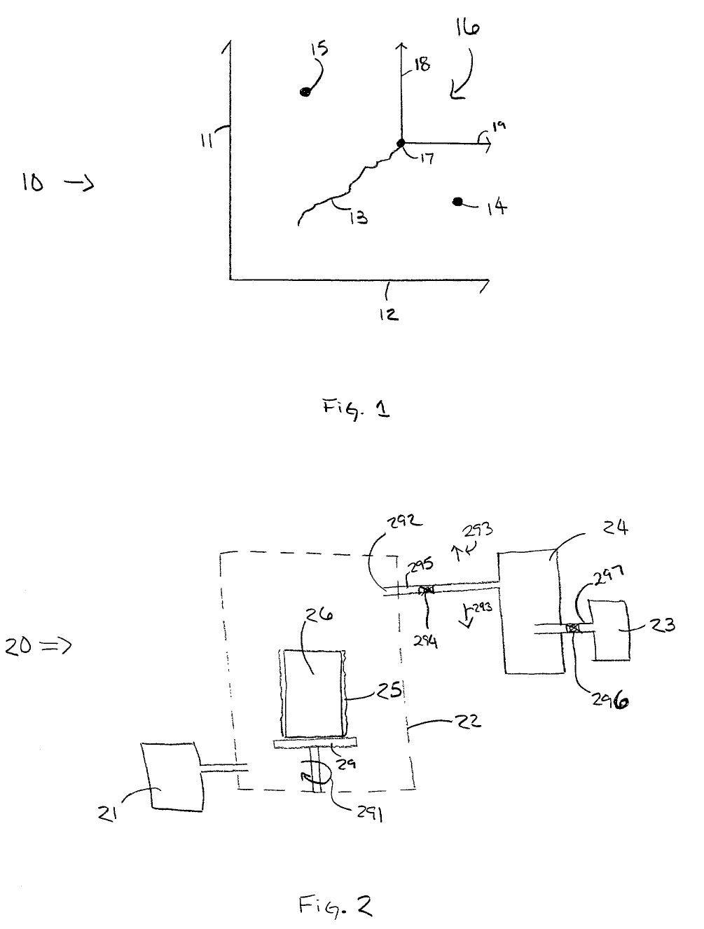

FIG. 2 is a side view of a coating system 20 for coating a medical device 26 as may be used in one embodiment of the present invention. In FIG. 2 a coating chamber 22 is shown in cutaway view. This coating chamber 22 may be made from numerous materials and may have numerous configurations. In this embodiment the coating chamber 22 may have a general cylindrical shape and may be made from a composite steel that is welded together and designed to resist the temperatures and pressures that may be required to carry out the coating procedures being performed within the chamber 22. The coating chamber 22 in this embodiment may contain a support 29 for holding the medical device 26 that may be coated in the chamber 22. This support 29 may have a top tray or basket and may be able to rotate, as indicated by arrow 291, in order to better coat the device placed thereupon. By rotating the support 29, the nozzle 292, of the supercritical fluid tank 24, may be able to better access all of the surfaces of the device 26 during the coating process. This nozzle 292 may be fluidly coupled, through a tube 295, to the supercritical tank 24. This supercritical tank 24 may be, in-turn, fluidly coupled to a therapeutic tank 23. In one embodiment, the nozzle 292 may be able to move up and down within a slot (which is not visible in this view) of the coating chamber 22 in order to be able to more directly reach and coat the device 26.

The supercritical fluid tank 24 may be used to store a material and bring it into its supercritical range as well as to store a material that has already been brought within its supercritical range. In either case the release of the supercritical fluid from tank 24 may be controlled by valve 294, which is disposed in tube 295. As necessary, during the coating process, valve 294 may be opened to allow supercritical fluid to flow from the tank 24 to the chamber 22.

A therapeutic tank 23 may also be in fluid communication with the supercritical tank 24. This therapeutic tank 23 may be used to store and release therapeutic into the supercritical tank 24 through line 297 as controlled by valve 296. Like the supercritical fluid tank 24, the therapeutic tank 23 may also accept therapeutic at predetermined pressure and temperature in addition to being configured to adjust the temperature and pressure of therapeutic placed within it. While the supercritical fluid tank 24 and the therapeutic tank 23 are shown to be cylindrical in shape they may be of various shapes and sizes and may be made from various materials. It is preferable, however, that they are compatible with the fluids and therapeutics that they may store and come in contact with during their use.

In use the medical device 26, which may be a stent, an aneurism coil or any other implantable medical device, may be placed onto the support 29 of the coating chamber 22. In this embodiment, the medical device 26 may be precoated with a swellable carrier coating such as the hydrocarbon based elastomeric polymer disclosed in U.S. Pat. No. 5,741,331, the disclosure of which is incorporated herein in its entirety by reference. This carrier coating may be used to accept and carry, through dissolution or some other suitable means, a therapeutic that comes in contact with the coating during the coating process. In this embodiment carbon dioxide may be used as the supercritical fluid and paclitaxel may be used as the therapeutic. The carbon dioxide may be placed within the supercritical tank 24 and may, then, be brought into its supercritical range by applying the requisite pressure and temperature. Once the carbon dioxide has reached its supercritical range, therapeutic stored in the therapeutic tank 23 may, then, be fed into the supercritical tank 24 and mixed with it. This may be done by opening the valve 296 in line 297 and injecting the therapeutic from tank 23 into tank 24. The operation of the valve 296, as well as the control of the other activities, may all be centrally controlled by a processor or may, alternatively, be controlled manually by an operator of the system 20 or some other suitable means.

After the requisite amount of therapeutic (in this example paclitaxel) has been injected and mixed with the supercritical carbon dioxide in the supercritical tank 24, the supercritical fluid, now carrying the therapeutic, may, then, be ejected out of the tank 24, through the nozzle 292, and into the chamber 22. In one embodiment the supercritical carbon dioxide and the therapeutic may be ejected above the device 26 and, thus, may act to fill the coating chamber 22 with a specific predetermined amount of fluid and therapeutic. In this embodiment, the amount of therapeutic resident within the chamber 22 may be measured by regulating the amount of time that the valve 294 is open or by measuring the amount of fluid that passes through the valve 294. These measuring techniques, as well as the others that may be used, may be performed so that the amount of therapeutic admitted into the chamber 22 is readily discernible.

The device 26, now sitting in a bath of therapeutic laden supercritical carbon dioxide, may absorb the therapeutic into its carrier coating 25, a coating which may have swelled after coming in contact with the supercritical carbon dioxide, thereby making it more receptive to accepting and carrying the therapeutic. Next, after a predetermined amount of time has passed, a valve to the recycling chamber 21 may be opened and a vacuum may be placed in the coating chamber 22 by the recycling chamber 21 to evacuate the unused supercritical carbon dioxide and therapeutic from the coating chamber 22. After entering the recycling chamber 21 the carbon dioxide may, then, be brought below its supercritical temperature and the unused therapeutic may be recaptured for later use. The entire process may, then, be repeated with another medical device.

In one embodiment the rate of release of the supercritical carbon dioxide from the chamber 22 and, thus, the depressurization of the supercritical carbon dioxide, may be controlled such that a foamed or porous morphology of the coating 25 is created. In this embodiment, by regulating the depressurization of the fluid, the surface area of the coating 25 can be increased in a controlled fashion. By adjusting the surface area of the coating 25 the rate of elution of any therapeutic contained therein can be adjusted. Alternatively, the porosity of the coating 25 can be modified to make it better tailored to accept and carry certain therapeutic agents including: cells, DNA, and proteins of both soluble and insoluble materials.

After the combined supercritical fluid and therapeutic have been removed from the coating chamber 22, the carrier coating 25, no longer exposed to the supercritical carbon dioxide, may shrink to its original size or at least close to its original size. This expansion and retraction may allow the coating to more efficiently carry a therapeutic to the target site as the therapeutic may fit within the interstices of the polymer during its expanded state than if the polymer had not swelled.

Alternatively, rather than having the tube 295 remain stationary during the coating process, it may also be moved up and down as indicated by arrows 293. In this alternative embodiment, the device 26, located on the support 29, may be concurrently rotated to expose its various surfaces to the nozzle 292 of the tube 295. Here, rather than bathing the device 26 in the supercritical carbon dioxide and therapeutic, the supercritical carbon dioxide and therapeutic may be ejected directly at the swellable polymer, through the nozzle 292, as the nozzle is moved up and down in the coating chamber 22. This alterative, direct spray configuration, may be useful in several situations including: if variable concentrations or patterns of the therapeutic are required on the surface of the device 26; if variable therapeutics are being ejected on the device 26 in alternating or otherwise patterned steps; and, if adequate pressures or temperatures can not be maintained in the coating chamber 22 such that the supercritical fluid leaving the nozzle 292 remains in its supercritical state for a short duration of time, thereby requiring that the fluid be immediately interfaced with the coating 25 in order to effectively transfer the therapeutic.

In addition to coating a single medical device as described above, more than one medical device may be placed into the coating chamber. One benefit of this alternative embodiment is that, as each of the devices are exposed to the same bath for the same period of time, the devices may then be grouped or used together as they may contain virtually the same amount of therapeutic.

While paclitaxel and carbon dioxide are described in some of the above embodiments, other combinations of therapeutics and supercritical fluids may also be employed. It is preferable, however, that the supercritical fluid and the chosen therapeutic be compatible with one another and with the carrier coating resident on the medical device.

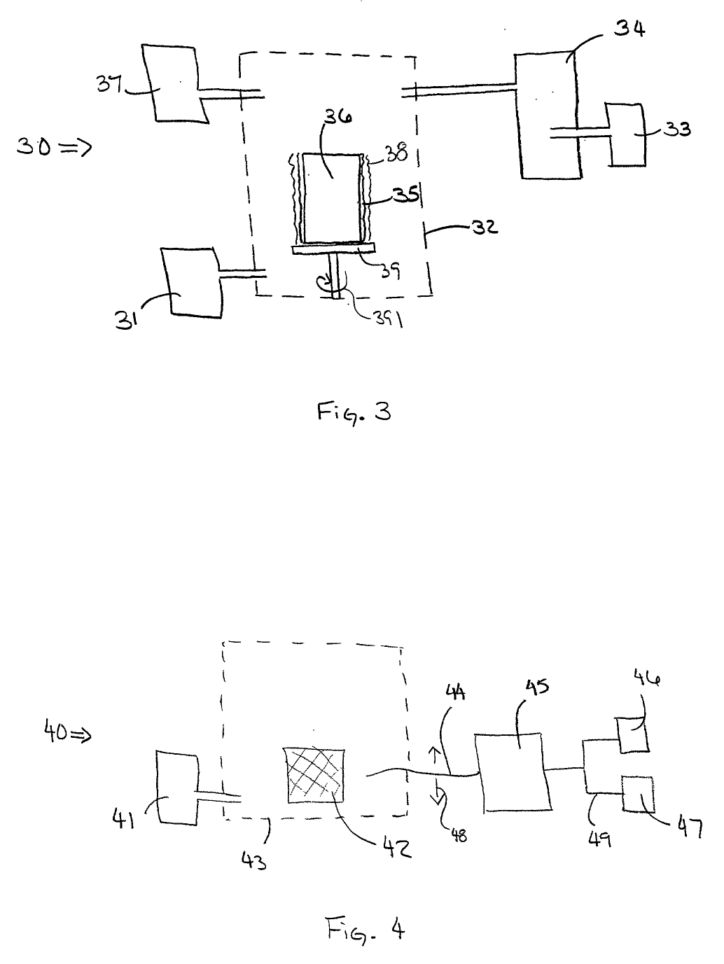

FIG. 3 is an alternative embodiment of the present invention. In FIG. 3 the coating system 30 contains a recycling chamber 31, a first supercritical fluid tank 34, a second supercritical tank 37, a therapeutic tank 33, a coating chamber 32, and a support 39, which may be rotatable in the direction of arrow 391. As can be seen, a stent 36 has been placed on the support 39 in this embodiment.

In use, after the stent 36 has been placed on the support 39, the second supercritical fluid tank 37 may be used to flood the coating chamber 32 with a supercritical fluid and, thus, swell the coating 35 resident on the surface of the stent 36. Then, after the coating 35 has swelled, the supercritical fluid resident within tank 34, which has been previously mixed with therapeutic from tank 33, may be released into the chamber 32. An advantage of swelling the coating before exposing it to the therapeutic is that the coating may be better able to receive the therapeutic due to its enlarged state. Coating line 38 illustrates the degree to which the original coating 35 may swell when exposed to certain supercritical fluids.

The recycling chamber 31 in this embodiment may not only be used to draw unused supercritical fluid from the coating chamber 32 it may also be used to increase the rate in which the supercritical fluid enters the chamber 32 by placing a vacuum force in the coating chamber 32 as the supercritical fluid enters the chamber 32. If a vacuum force is used, it is preferable that the pressure maintained in the first and second supercritical tanks be adjusted to compensate for the vacuum forces placed on the coating chamber 32. This is done so that when supercritical fluid is released from the supercritical tanks it does not drop below its critical pressure or temperature and lose some or all of its desired properties. As in the above embodiments, the release of the supercritical fluid as well as the other required processes may be controlled manually by an operator of the system and may also be controlled by a processor (or some other apparatus) that monitors the temperatures, pressures, and other variables of the system.

FIG. 4 is another alternative embodiment of the present invention. As is evident in the system 40 of FIG. 4 the stent 42 does not rest on a rotatable platform within the coating chamber 43 as in the previous embodiment but, rather, rests directly on the floor of the coating chamber 43. In this embodiment, contrary to the above described embodiments, the stent has not been precoated with a carrier coating. Instead, the coating may be applied with the supercritical fluid at the same time as the therapeutic or alternatively, before the supercritical fluid and therapeutic is applied. For example, in the system of FIG. 4, a coating tank 47 may be employed and used to spray a coating onto the stent after the stent has been placed into the chamber 43 but before the stent 42 is interfaced with a supercritical fluid carrying the therapeutic.

The therapeutic tank 46 may be in fluid communication with both the supercritical tank 45 and the coating tank 47 via line 49 in this embodiment. Alternatively, in another embodiment employing the system 40 of FIG. 4, the therapeutic and the coating may be premixed before being drawn into the supercritical fluid tank 45 and may, then, interface with a supercritical fluid contained therein. Once the coating and the therapeutic have been mixed with the supercritical fluid resident within the supercritical fluid tank 45 the entire mixture may, then, be ejected, through line 44, at the stent 42 resident within the chamber. The line 44 in this embodiment may be long enough to reach completely around the stent in order to directly coat the device. The line 44 may be manipulated by a user of the system or may, alternatively, be controlled by some mechanical means.

In this embodiment, as the coating chamber 43 may not be maintained at the critical pressure and temperature of the fluid, the end of the line 44 may be placed in close proximity to the stent during the coating process, such that the supercritical fluid being ejected from the line 44 remains in its supercritical range until it reaches the stent 42. After the coating and therapeutic have been applied the recycling chamber 41 may then be used to evacuate the coating chamber 43 and store the used mixture for later use.

Alternatively, and as mentioned above, rather than mixing the coating and the therapeutic prior to it being injected into the supercritical tank, the coating may be mixed with a supercritical fluid and then applied to the medical device. Furthermore, in another embodiment, the medical device may be directly covered with a therapeutic without the use of a carrier coating. Thus, multiple coating scenarios are plausible within the spirit and scope of the present invention.

While a single stent has been described in some of the above embodiments other medical devices may also be coated using each of these various embodiments. The range of medical devices that may be coated include: expandable and self expanding stents, balloon catheters, vena-cava filters, aneurysm coils, stent-grafts, a-v shunts, angio-catheters, and PICC's. Moreover, in addition to using paclitaxel as the therapeutic the above invention may also be employed with a wide variety of other therapeutics, which include, for example: pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1(“VP22”)), and viral, liposomes and cationic and anionic polymers and neutral polymers that are selected from a number of types depending on the desired application. Non-limiting examples of virus vectors or vectors derived from viral sources include adenoviral vectors, herpes simplex vectors, papilloma vectors, adeno-associated vectors, retroviral vectors, and the like. Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; agents blocking smooth muscle cell proliferation such as rapamycin, angiopeptin, and monoclonal antibodies capable of blocking smooth muscle cell proliferation; anti-inflammatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitorfurantoin; anesthetic agents such as lidocaine, bupivacaine, and ropivacaine; nitric oxide (NO) donors such as lisidomine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warafin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogeneus vascoactive mechanisms; survival genes which protect against cell death, such as anti-apoptotic Bcl-2 family factors and Akt kinase; and combinations thereof. Cells can be of human origin (autologous or allogenic) or from an animal source (xenogeneic), genetically engineered if desired to deliver proteins of interest at the injection site. The delivery mediated is formulated as needed to maintain cell function and viability. Any modifications are routinely made by one skilled in the art.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides of the invention can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof. Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

These therapeutic agents can be used, for example, in any application for treating, preventing, or otherwise affecting the course of a disease or tissue or organ dysfunction. For example, the methods of the invention can be used to induce or inhibit angiogenesis, as desired, to prevent or treat restenosis, to treat a cardiomyopathy or other dysfunction of the heart, for treating Parkinson's disease or a stroke or other dysfunction of the brain, for treating cystic fibrosis or other dysfunction of the lung, for treating or inhibiting malignant cell proliferation, for treating any malignancy, and for inducing nerve, blood vessel or tissue regeneration in a particular tissue or organ.

Using supercritical fluids to deposit a therapeutic on or in a medical device is described herein. While several embodiments are presented it should be appreciated that other embodiments, modifications, and variations of the present invention are also plausible and may be made without departing from the spirit and scope of the present invention.

What is claimed is:

1. A method of coating a medical device comprising:
interfacing a therapeutic with a supercritical fluid; and
transferring the therapeutic from the supercritical fluid to the medical device.

2. The method of claim 1 further comprising:
applying a carrier coating to the medical device.

3. The method of claim 1 wherein transferring the therapeutic from the supercritical fluid to the medical device includes spraying the supercritical fluid at the medical device.

4. The method of claim 1 wherein transferring the therapeutic from the supercritical fluid to the medical device includes exposing the medical device to a bath of supercritical fluid.

5. The method of claim 1 wherein the therapeutic substantially dissolves in the supercritical fluid upon.

6. The method of claim 1 wherein the therapeutic is colloidally suspended in the supercritical fluid.

7. The method of claim 1, further comprising:
applying a vacuum force to a chamber containing the medical device.

8. The method of claim 1 wherein the therapeutic is combined with a carrier coating.

9. The method of claim 1 further comprising:
collecting the supercritical fluid after transferring the therapeutic from the supercritical fluid to the medical device; and
removing residual therapeutic from the supercritical fluid after collecting the supercritical fluid.

10. The method of claim 1 wherein the supercritical fluid is supercritical carbon dioxide and the therapeutic is paclitaxel.

11. The method of claim 1 wherein the medical device is chosen from a group consisting of a stent, a peripherally inserted central catheter, an angio-catheter, a stent-graft, a vena-cava filter, and an aneurysm coil.

12. A method of coating a medical device comprising:
placing a medical device in a coating chamber;
coating the medical device;
interfacing a therapeutic with a supercritical fluid; and
exposing the coating to the supercritical fluid.

13. The method of claim 12 wherein exposing the coating to the supercritical fluid includes spraying the supercritical fluid at the medical device.

14. The method of claim 12 wherein exposing the coating to the supercritical fluid includes flooding the coating chamber with the supercritical fluid after the therapeutic has been interfaced with the supercritical fluid.

15. The method of claim 12 further comprising:
swelling the coating by exposing the coating to a supercritical fluid.

16. An article of manufacture comprising:
a medical device having a releasable therapeutic on one of its surfaces,
the medical device manufactured by:
placing the medical device in a coating chamber, and
exposing the medical device to a supercritical fluid carrying a therapeutic.

17. The article of manufacture of claim 16 wherein the medical device is chosen from a group consisting of: a stent, a catheter, a vena cava filter, an aneurysm coil, a stent-graft, an a-v shunt, an angio-catheter, and a peripherally inserted central catheter.

18. The article of manufacture of claim 16 wherein the manufacture of the medical device also comprises coating the medical device with a coating and swelling the coating on the medical device after it is applied to the medical device.

19. The article of manufacture at claim 16 wherein the supercritical fluid exposed to the medical device contains a coating polymer.

20. A system of coating a medical device comprising:
a supercritical fluid tank;
a therapeutic tank; and
a coating chamber,
the coating chamber in communication with the supercritical fluid tank,
the therapeutic tank in communication with the supercritical fluid tank,
the supercritical fluid tank containing supercritical fluid.
